(19) United States
(12) Patent Application Publication
(10) Pub. No.: US 2019/0274972 A1
(43) Pub. Date: Sep. 12, 2019

(54) CBD Alkaline Water With Fulvic Acid

(71) Applicant: Michael Ochoa, Phoenix, AZ (US)

(72) Inventor: Michael Ochoa, Phoenix, AZ (US)

(21) Appl. No.: 15/919,156

(22) Filed: Mar. 12, 2018

(51) Int. Cl. A61K 31/05; A61K 35/10

(52) U.S. Cl. CPC A61K 31/05; A01H 5/00; A61K 35/10

(57) ABSTRACT

Disclosed herein is an alkaline water solution including CBD oil mixed homogeneously with the alkaline water. In a second exemplary embodiment, Fulvic Acid is added to the solution of CBD oil and alkaline water. In general, the CBD oil makes up no more than 3% of the alkaline water solution and no more than 2000 mg in a 15 ounce container. In the solution including Fulvic Acid, a 15 ounce solution contains between 5 mg and 15 mg.

### BACKGROUND OF THE INVENTION

### Field of the Invention

The disclosure herein relates to water which is healthy for human consumption. More specifically, the water herein meant for human consumption has a pH in the alkaline range and includes CBD and in other exemplary embodiments, Fulvic Acid.

### Background

As is well known, medical cannabis or marijuana has now entered into mainstream, including conventional Western medicine. For many years it has been known, that medical cannabis is effective in relieving inflammatory conditions such as arthritis. It is also widely known, that medical cannabis has also been effective in relieving aches, pains, including headaches. What is not widely known is that medical marijuana is effective in treating mental health ailments such as depression, anxiety and chronic stress caused from living a in our industrial society.

Cannabidiol abbreviated as CBD is one of at least 113 active cannabinoids identified in cannabis. CBD is known as a phytocannabinoid and accounts for roughly 40% of the plant's extract. CBD does not have any psychoactive effects, unlike tetrahydrocannabinol (THC). In fact, it is widely believed that CBD has a markedly positive impact on disordered thinking and especially on anxiety.

Unlike smoking medical marijuana, CBD water is safe and effective without psychotropic effects. Among doctors and other health professionals, CBD water is widely recommended to relieve chronic ailments such as inflammation, pain and anxiety, as noted above. And, while TF-IC may provide harmful psychotropic effects, CBD water is especially useful because it provides patients with a simple, non-detectable and harmless way to medicate themselves. The fact that CBD is harmless is not to be underestimated. As anyone familiar with chronic pain and inflammation is aware, overmedication can readily occur, for example opiate addiction has become one of the leading causes of medical issues in the world. Since CBD is harmless, safe and effective, it may readily replace opiates without the unwanted side effects and in fact provide more effective relief since it is widely known that CBD actually recruits the body's own functions to work. And, additionally, CBD is known as a treatment for opiate addiction.

CBD water not only hydrates but provides athletes with the means of relieving their chronic ailments such as, inflammation, pain and anxiety disorders. And, this can all be done without detection or violating various institutional rules against the use of cannabis. As anyone familiar with high-level amateur or professional athletics knows minimizing the inflammation, pain, and stress placed on the body enables an athlete to achieve his personal best performance while minimizing the risk of injury. Quite clearly, this leads the athlete to a longer, healthier and more prosperous career.

Of course, it is not without difficulties to combine CBD with water. As anyone knows, CBD is an oil and as such CBD doesn't mix well with water. CBD oils and tinctures added to water may do some good, but given that water and oil don't mix, the effectiveness of such mixtures is thought to be quite limited.

CBD water is typically water infused with CBD. CBD compounds are typically broken down and an attempt is made to evenly distribute them in the water. This is done in an effort to make the CBD have a greater bioavailability so that the CBD penetrates the digestive tract and the bran-blood barrier more effectively. It is widely known that as much as 90% of the CBD can be lost through poor bioavailability.

Although CBD is a cannabinoid like THC, CBD is non-psychoactive and does not have the psychotropic effects associated with THC. CBD's ability to alleviate symptoms such as muscle pain, joint inflammation, nausea, anxiety, and other ailments have made it a valuable medical resource with treating cancer, epilepsy, MS, PTSD, opiate addiction, and more. With ease and comfort, CBD Water is capable of putting your body into an overall sense of calm and relaxation. Muscle pain and discomfort is quickly alleviated, allowing your body to heal and recover.

Fulvic Acid

It is general knowledge that in recent decades, soil minerals have become depleted in our agricultural lands. As a result of this, the nutrient value of our food has markedly declined. In the USA, there are record amounts of solid plastic waste, open chemical dumps, all resulting in lower soil nutrient levels. It is becoming increasingly more difficult to find nutrient dense food. It is questionable whether it is even possible to get enough nutrients solely from food at this time.

In the last 100 years, over farming, mono-cropping and the use of chemical pesticides and artificial fertilizers in commercial, large scale agriculture has depleted the soil of minerals to dangerously low levels. Since it is no longer possible obtain one's food from pristine soils, since mass farming does not re-mineralize the soil. In earlier days, it was common practice for farmers to carefully replenish the soil nutrient levels with compost, crop rotation and soil rest intervals. Now, mass farming bypasses such composting processes and instead employs use of chemical based fertilizers containing neuro-toxins allowing crops to grow, but depleting and poisoning the soil in place of replenishment. Studies have shown that over the past 100 years, the mineral levels in agricultural soils have drastically dropped on over by 70% in Europe, 75% in Asia, and 80% in North America.

Trace minerals in the soil are perhaps the most to suffer depletion. Such trace minerals are essential to one's health. Trace minerals work synergistically with other minerals, vitamins, enzymes, and amino acids in a wide variety of biochemical reactions. These minerals are especially critical for preventing neuro-muscular diseases, such as MD, MS and ALS. The increase of such diseases has grown astoundingly in recent years.

What is therefore needed is a more effective delivery system for delivering the CBD to the body to achieve the beneficial effects of the CBD without the harmful psychotropic effects of THC. What is also needed is a way to combine CBD water with other elements to body function.

### SUMMARY OF THE INVENTION

CBD alkaline water is alkaline water infused with CBD. In one method of combining CBD with water, CBD compounds are broken down into nanoparticles and evenly distributed in the water; effectively homogenizing the mixture. The result is water with a bioavailable source of CBD that is able to penetrate tissue like the digestive tract and blood-brain barrier much more effectively. Typically, medicinal CBD is consumed through edibles. In this process, much of the CBD can be lost along the way (sometimes as much as 90%) due to its poor bioavailability.

This method can be adapted within the spirit and scope of the present invention. However, the preferred method is to use proprietary CBD oil which has been found to mix readily with alkaline water. However, either method of combining CBD with water is within the spirit and scope of this invention.

Fulvic acid is a natural substance found in hummus that typically grows near water and is a water-soluble substance. Full like acid is produced by decomposing organic materials in the ground. The plant-derived material is one of many organic substances that make up Fulvic Acid. Plants absorb the substance, making the vitamins and minerals in the plants more absorbable. Because of the damage caused to the natural growing environment, people take supplements that contain Fulvic acid to make up for the lack of the organic substance in the soil.

Fulvic acid plays a role in protecting your cells from oxidation that occurs from environmental toxins. It makes nutrients more soluble and available for use by the cells in your body. Fulvic acid is an antioxidant that fights off the damage incurred from free radicals that enter your body through the food you eat and through outside environmental toxins. It is an electrolyte that helps balance the minerals that drive neurons in your brain. Fulvic acid also plays a role in RNA and DNA synthesis that might result in serious consequences if used in large doses. However, when used in the quantities as set forth in this disclosure, Fulvic Acid is both safe and effective for combating disease and for maintaining health.

In accordance with the above objects and those that will be mentioned and will become apparent below, the CBD alkaline water mixture in accordance with this disclosure comprises:
alkaline water having a pH greater than 6.5 and CBD in solution with the alkaline water, wherein the CBD is greater than 3% and less than 2000 mg per 15 ounces of solution.

In another exemplary embodiment, the CBD alkaline water mixture in accordance with this disclosure comprises:
alkaline water having a pH greater than 6.5 and CBD in solution with the alkaline water, wherein the CBD is greater than 3% and less than 2000 mg per 15 ounces of solution; and
5 mg to 15 mg of Fulvic Acid in 15 ounces of alkaline water.

### DETAILED DESCRIPTION OF THE DISCLOSURE

For a further understanding of the objects and advantages of the present disclosure, reference should be had to the following detailed description, where the invention will now be described.

The disclosure herein describes a mixture of the CBD in alkaline water. As noted above, CBD is an oil and does not readily mixes with water. A proprietary formulation of CBD with proprietary alkaline water allows the CBD to fully next and become homogeneous throughout the solution. After the appropriate amounts of CBD are added to the alkaline water with CBD, Fulvic acid is added in the appropriate quantities in exemplary embodiments.

Mixture 1

In the first exemplary embodiment, the solution begins with alkaline water. As is well known, alkalinity is based on a pH scale. The scale goes from won to 14. A pH of 1 is the most acidic and a pH of 14 is the most alkaline. In the exemplary embodiment of mixture 1, the alkaline water has a pH greater than 6.5 and preferably over 7.

A proprietary CBD oil is then added to the alkaline water and mixed to achieve a homogeneous solution. After the addition of the CBD, the alkalinity of the water is checked. Should the alkalinity fall below a pH of 6.5, additional alkaline water is added to the solution until the solution achieves the appropriate pH level.

Once again the solution is checked, but this time for CBD level. The desired CBD level is 3% to as much as 2000 mg. per 15 ounces. In fact, all measurements described above are based upon 15 ounces of total liquid.

CBD is added until the desired result is achieved. The CBD level is then checked. Upon achieving the desired CBD level, the alkalinity of the solution is checked again and the process is repeated until both the desired level of CBD and the desired level of alkalinity is achieved.

Mixture 2

In the exemplary example of Mixture 2, the range is narrowed so that the alkalinity of the solution is greater than a pH of 7.0. The process described above is repeated until the desired result is achieved.

Mixture 3

In mixture 3, the range is again narrowed so that the alkalinity of the solution is greater than a pH of 7.1. The process described above is repeated until the desired result is achieved.

In additional exemplary embodiments, the range is set as desired and may even be set to a specific level. The process described above is repeated until the desired result is achieved.

Mixture 4

In other exemplary embodiments, the solution described in the various above exemplary embodiments is mixed with Fulvic Acid. In Mixture 4, the exemplary embodiment includes 5 mg to 15 mg of Fulvic Acid. Since of Fulvic Acid is not an oil—like CBD, the same issues associated with mixing CBD and alkaline water are not present.

However, the same methodology described above is repeated with regard to the addition of Fulvic Acid. Alkaline water and Fulvic Acid are added and tested to make sure that the desired levels are achieved.

Mixture 5

In the exemplary example of Mixture 5, the range of the Fulvic Acid is narrowed so that the Fulvic Acid of the solution is between 7 and 15 g per 15 fluid ounces. In order to achieve the desired levels of Fulvic add and alkalinity the process described above is repeated until the desired result is achieved.

Mixture 6

In the exemplary example of Mixture 6, the range of the Fulvic Acid is narrowed so that the Fulvic Acid of the solution is between 10 and 15 g per 15 fluid ounces. In order to achieve the desired levels of Fulvic Acid and alkalinity the process described above is repeated until the desired result is achieved.

Mixture 7

In the exemplary example of Mixture 5, the range of the Fulvic Acid is narrowed so that the Fulvic Acid of the solution is between 10 and 15 g per 15 fluid ounces. In order to achieve the desired levels of Fulvic Acid and alkalinity the process described above is repeated until the desired result is achieved.

While the foregoing detailed description has described several embodiments of alkaline CBD water in accordance with this disclosure, it is to be understood that the above description is illustrative only and not limiting of the disclosed invention. Particularly, the alkaline CBD water may include a virtual infinite number of solution combinations with and without Fulvic Acid. Thus, the disclosure is to be limited only by the claims as set forth below.

1. A solution of cannabidiol (CBD) and alkaline water, comprising:
alkaline water having a pH of greater than 6.5 and less than 9; and
CBD rendered into nanoparticles and homogenized with the alkaline water, wherein the amount of CBD is not less than 3% of the mixture and not more than 2000 mg per 15 ounces of alkaline water.

2. The solution of CBD and alkaline water as set forth in claim 1, wherein the alkaline water has a pH of greater than 6.8.

3. The solution of CBD and alkaline water as set forth in claim 1, wherein the alkaline water has a pH of greater than 7.0.

4. The solution of CBD and alkaline water as set forth in claim 1, wherein the alkaline water has a pH of greater than 7.1.

5. The solution of CBD and alkaline water as set forth in claim 1, wherein the CBD in 15 ounces of alkaline water solution is greater than 5 mg.

6. The solution of CBD and alkaline water as set forth in claim 1, wherein the CBD in 15 ounces of alkaline water solution is greater than 10 mg.

7. The solution of CBD and alkaline water as set forth in claim 1, wherein the CBD in 15 ounces of alkaline water solution is greater than 12 mg.

8. A solution of CBD and alkaline water, comprising:
alkaline water having a pH of greater than 6.5;
CBD rendered into nanoparticles and homogenized with the alkaline water, wherein the amount of CBD is not less than 3% of the mixture and not more than 2000 mg per 15 ounces of alkaline water; and
Fulvic Acid in solution with the alkaline water and CBD, wherein the amount of Fulvic Acid in 15 ounces of alkaline water solution is between 5 mg and 15 mg.

9. The solution as set forth in claim 8, wherein the amount of Fulvic Acid is between 10 and 15 mg.

10. The solution as set forth in claim 8, wherein the amount of Fulvic Acid is between 12 and 15 mg.

11. A method for rendering cannabidiol (CBD) enabling maximum bioavailability including the steps of:
providing less than 15 ounces alkaline water above 6.5 pH and below 9 pH;
a CBD oil rendered in a nanoparticle form and is added at a concentration not to exceed 3% to the alkaline water creating a homogenous solution; and
determining the pH level of the homogenous solution, and if it is below 6.5 pH, alkaline water is added, and if the homogenous solution is above 9 pH, Fulvic Acid is introduced until a pH reading is achieved below 9 pH.
